(19) United States
(12) Patent Application Publication
(10) Pub. No.: US 2020/0306172 A1
(43) Pub. Date: Oct. 1, 2020

(54) DEPIGMENTING DERMATOLOGICAL AND COSMETIC COMPOSITIONS

(71) Applicant: NUNII LABORATOIRE, Grasse (FR)

(72) Inventors: Francois Montcriol, Villeneuve-Loubet (FR); Niamh Cogan, Valbonne (FR)

(21) Appl. No.: 16/483,919

(22) PCT Filed: Apr. 13, 2017
(86) PCT No.: PCT/FR2017/050890

§ 371 Date: Feb. 18, 2020

(30) Foreign Application Priority Data
Feb. 6, 2017 (FR) 1750981

(51) Int. Cl. A61K 8/9789; A61K 8/44; A61K 8/34; A61K 8/67; A61K 8/60; A61K 8/04

(52) U.S. Cl. CPC A61K 8/9789; A61K 8/44; A61K 8/347; A61Q 19/02; A61K 8/602; A61K 8/676; A61K 8/042; A61K 8/675

(57) ABSTRACT

The invention concerns dermatological or cosmetic compositions having a depigmenting action by topical application and comprising at least an effective amount of the specific combination of six constituents. It also concerns the use of an effective amount of said specific combination of six constituents acting in combination on melanogenesis as active compounds for the preparation of a dermatological composition intended for treating pathological or accidental hyperpigmentation. Finally, it concerns a cosmetic treatment method intended to improve the aesthetic appearance of the skin, consisting of applying a sufficient amount of a composition according to the invention to the skin.

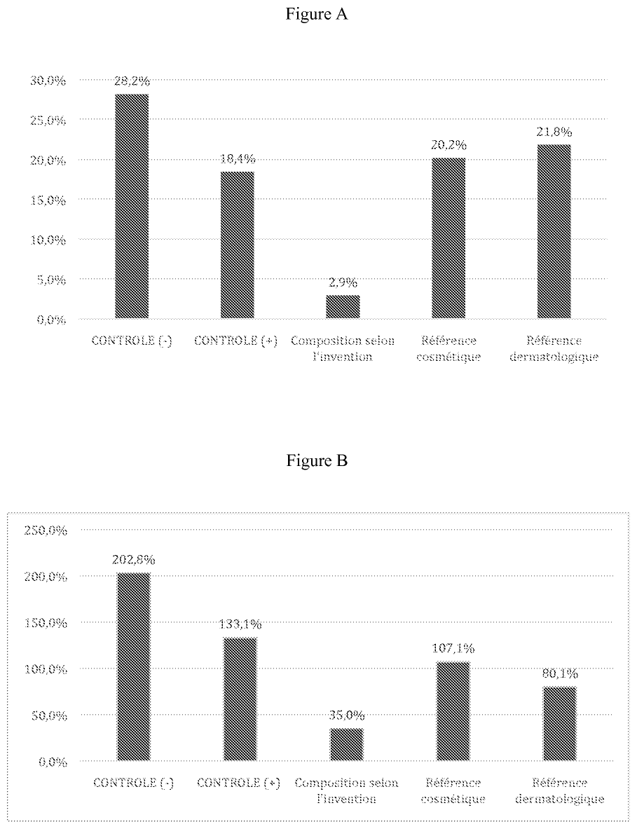

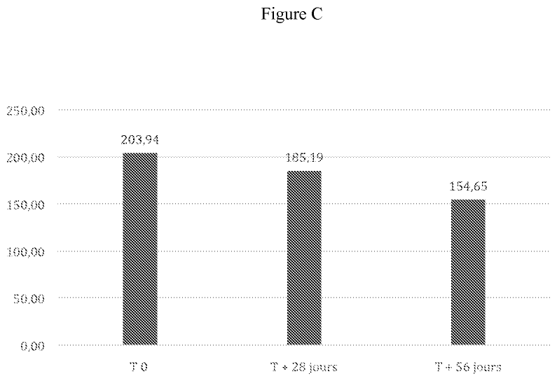

This invention concerns a dermatological or cosmetic composition having a depigmenting action containing, as an active substance, a combination of six depigmenting compounds.

Depigmentation of the skin may be desired in various circumstances, either as a cosmetic treatment to obtain an overall lightening of the skin or to eliminate or reduce marks caused by local pigmentation disorders, or as a dermatological treatment to treat hyperpigmentations.

The skin comprises several integrated layers, from the surface layer, the epidermis (epithelial tissue), to the deeper layers, the dermis and the hypodermis (connective tissue), and each has specific properties enabling the skin as a whole to respond and adapt to the conditions of its environment.

Pigmentation of the skin is due to the presence of melanin in the epidermis and dermis. Melanin is produced by the melanocytes located primarily in the basal layer, in the presence of tyrosinase (or monophenol monooxygenase), a copper-protein enzyme which catalyzes the conversion of tyrosine to dihydroxyphenylalanine (dopa), which is subsequently converted into dopaquinone and then dopachrome, leading to melanin according to a complex mechanism. This biosynthesis, or melanogenesis, is a complex process which is relatively well known today.

There are pathologies associated with malfunction of the melanocytes which cause hyperpigmentation, such as age spots (actinic lentigo) or melasma. These pigment defects do not endanger the lives of sufferers but have an important psychological impact.

Actinic lentigo (senile lentigo, age spots) is a common acquired hyperpigmentation characterized by small brown spots on the areas exposed to light (face, back, hand and arm). It can be considered to be a sign of aging of the skin caused by excessive exposure to ultraviolet rays of sunlight. From the histological point of view, actinic lentigo is characterized by an increase in melanin load, the number of melanocytes and the surface of the epidermis (hyperplasia of the interpapillary ridges).

Melasma, often called “mask of pregnancy”, is a common acquired hyperpigmentation characterized by brown spots on the face which affects 90% of pregnant women. The triggering factors are pregnancy, hormonal contraception and exposure to the sun. However, the pathogenesis of melasma is not yet well known. Melasma is caused by increased melanin deposition in the epidermis. The melanocytes in the affected skin are larger, more dendritic, and contain more melanosomes in the melanocytes of the affected skin, which suggests that the melanocytes are more active and more differentiated in the lesional areas of melasma. We also note an increase in the number of melanocytes in the hyperpigmented areas.

Another known hyperpigmentation of the skin is post-inflammatory hyperpigmentation. This hyperpigmentation is induced by a skin lesion or a skin inflammation of exogenous origin following a cosmetic procedure such as dermabrasion, laser treatments and chemical abrasion, or of endogenous origin such as skin diseases. This risk of pigmentary change is more widespread in Fitzpatrick phototypes IV, V and VI (Hispanic, Asian and African respectively). It is characterized by unsightly marks.

Many products are currently available on the market to remedy these problems of hyperpigmentation. Most of these products contain in their formulas one or more now controversial ingredients responsible for adverse effects. Indeed, these products contain ingredients whose safety is severely challenged by the health authorities in different countries or communities.

For example, according to the Directorate General for Competition, Consumer Affairs and Fraud Prevention (see http://www.economie.gouv.fr/dgccrf/Publications/Vie-pratique/Fiches-pratiques/Produits-de-blanchiment-de-la-peau), some depigmentation products, mainly used by women, pose significant health risks for these women. Thus, in nearly 60% to 70% of cases, these practices cause harmful effects for the skin, generating disorders of varying severity ranging from minor burns to the appearance of eczema, acne, irreversible stretch marks, excessive hair growth or frequent cases of structural fragility of the skin causing difficulty in healing.

The main causes of these adverse effects are the use in cosmetic formulas of ingredients which are either prohibited or restricted in their quantities by European and international cosmetic regulations.

Such is the case with hydroquinone, which has been prohibited for use in depigmenting cosmetic compositions in Europe since 2001 because this molecule is known to be cytotoxic and was classified as a category 2 CMR (carcinogenic, mutagenic, reprotoxic) substance liable to cause cancer. We are also familiar with the Kligman Trio depigmenting dermatological preparation to treat hyperpigmentation which comprises retinoic acid in 0.2% solution (6.0 ml), hydroquinone 5% (2.0 g) and hydrocortisone 1% (0.5 g). This preparation should be made only on prescription, with an average hydroquinone content of 4 to 5%. The prescribed amounts must imperatively be complied with, because the active ingredients are very aggressive for the skin, and the powders must be perfectly solubilized to avoid having any microcrystal in direct contact with the skin, to avoid irritation of the skin caused by Vitamin A acid inducing hypervitaminosis A.

This is also the case with kojic acid which is now banned for cosmetic use by the authorities in Switzerland (RS 817.023.31 DFI Order on cosmetics Annex 4—2015). It is considered that use at 1% is safe for the consumer, but over large areas of skin or when the skin barrier is impaired, as is the case after an exfoliation, the use of kojic acid endangers the health of consumers (Scientific Committee on Consumer Products, SCCP/1481/12, 22.5.12). The expert panel of the CIR (Cosmetic Ingredient Review) in its final report (Burnett C L et al. Final Report of the Safety Assessment of Kojic Acid as Used in Cosmetics, International Journal of Toxicology 2010; 29 (Supplement 4) 244S-273S) on the use of kojic acid in cosmetics states that at 1% there is no depigmenting activity of kojic acid and only a 4% dose shows a depigmenting effect.

Mention may also be made of azelaic acid which is also heavily used in cosmetics in most countries but whose cosmetic use is prohibited in all countries in South East Asia (ASEAN), as indicated in Annex II Part 1 of the ASEAN cosmetic directive, and in Switzerland (RS 817.023.31 DFI Order on cosmetics Annex 4—2015).

Therefore, there is indeed a need for dermatological and cosmetic compositions having depigmenting activity which exclude the use of hydroquinone, kojic acid and azelaic acid and which act on all the steps upstream and downstream from tyrosinase while guaranteeing very good efficacy and good tolerance.

Consequently, the problem which this invention proposes to solve is to develop new dermatological and cosmetic compositions having depigmenting properties, particularly topical compositions which can have effective depigmentation effects on the skin while presenting good tolerance.

The inventors of the present invention have developed new dermatological and cosmetic compositions with a particular combination of six components acting in combination on melanogenesis and whose effective amounts used yield better results than known compositions of the prior art in terms of efficacy while avoiding the side effects cited for said compositions of the prior art.

The present invention therefore concerns a dermatological or cosmetic composition having a depigmenting action by topical application and comprising at least an effective amount of a particular combination of the following six components acting in combination on melanogenesis:

a) a root extract of Glycirrizha glabra,

b) undecylenoyl phenylalanine,

c) resveratrol,

d) niacinamide,

e) α-arbutin,

f) 3-O ethyl-L-ascorbic acid,

in a carrier suitable for such an application.

In this description, unless otherwise specified in the text, it is understood that:
when a range is given, it includes the upper and lower limits of said range as well as all the numerical values between the limits of the range.
percentages are expressed by weight in the text, unless indicated otherwise.

In the present invention:
“topical application” means applying or spreading the compositions according to the invention on the surface of the skin,
“cosmetically or dermatologically acceptable” means that the composition according to the invention is suitable for contact with the skin without causing toxic reactions or intolerance,
“effective amount” and “sufficient amount” mean the necessary quantities either of the components or of the composition which need to be used to obtain effective depigmentation on the skin without causing any adverse effect,
“the particular combination of the six components acting in combination on melanogenesis” means that only the six components cited act on melanogenesis by a combined depigmenting activity,
“regional hyperpigmentations” means idiopathic melasmas occurring during pregnancy (“mask of pregnancy” or chloasma) or melasmas secondary to estrogen-progesterone contraception, cases of localized hyperpigmentation caused by hyperactivity and benign melanocytic proliferation, such as senile pigmentary spots, known as actinic lentigos,
“accidental hyperpigmentations” means photosensitization and post-lesion scarring,
“cosmetic treatment” means depigmentation of the skin for the following purposes: homogenization of the complexion, attenuation of spots and skin lightening.

The components of the compositions according to the invention are described in more detail below.

The root extract of Glycirrizha glabra (INCI nomenclature: Glycyrrhiza glabra (licorice) root extract) is marketed by MARUZEN PHARMACEUTICALS CO. LTD under the name LICORICE EXTRACT P-T(40) and contains 40% Glabridin, the principal ingredient of the hydrophobic portion corresponding to the following formula:

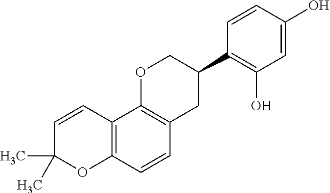 [SMILES: CC1(C)C=Cc2c(ccc3c2OC[C@@H](c2ccc(O)cc2O)C3)O1]

Glabridin is a flavonoid. It has clarifying properties by decreasing the activity of tyrosinase and demonstrates anti-inflammatory activity (Yokota T et al. Pigment Cell Res. 1998 December; 11(6):335-61). The product LICORICE EXTRACT P-T(40) is a preservative-free light yellow powder soluble in propylene glycol.

Undecylenoyl phenylalanine or undecylenoylphenylalanine (INCI nomenclature: undecylenoylphenylalanine) is marketed by SEPPIC under the brand name SEPIWHITE MSH®. Undecylenoylphenylalanine is an amphiphilic lipoamino acid which is a phenylalanine biovector. It is a pure compound which corresponds to the following formula:

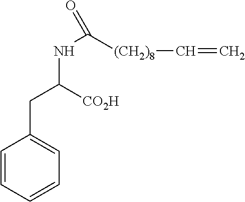

Undecylenoylphenylalanine is an antagonist of α-MSH, which gives it lightening properties. It comes in lipoamino acid form and has high skin affinity. It is easy to incorporate in depigmenting formulations, incorporable in any type of formula, completely stable, colorless and lightly scented.

Trans-resveratrol (INCI nomenclature: RESVERATROL), whose trade name is REGUFADE®, is marketed by DSM and corresponds to the following formula:

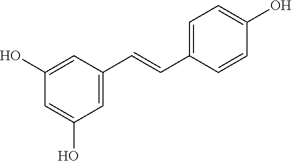 [SMILES: Oc1ccc(/C=C/c2cc(O)cc(O)c2)cc1]

Trans-resveratrol is of particular interest. It is part of the family of polyphenols with anti-free radical properties. This molecule has been the subject of numerous studies on these antioxidant properties. More recently, studies on melanogenesis have shown its lightening potential thanks firstly to its ability to interfere in the tyrosinase maturation stages, thus making the tyrosinases synthesized de novo ineffective (Newton R A et al, J. Invest. Dermatol. 2007 September; 127(9):2216-27) and secondly its ability to reduce the activity of the transcription factor MITF (Lin, C. B., Babiarz, L., Liebel, F. et al. J. Invest. Dermatol. 119:1330-1340,2002). Trans-resveratrol takes the form of a preservative-free 99% pure powder which is pale grey to amber in color and soluble in polyethylene glycols and propylene glycols.

Niacinamide or vitamin B3 (INCI nomenclature: niacinamide) marketed by DSM under the name Niacinamide PC corresponds to the following formula:

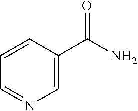

Niacinamide PC has clarifying properties through inhibiting the transfer of melanosomes from melanocytes to keratinocytes. (Hakozaki T et al. J. Dermatol. 2002 July; 147(1):20-31.) Niacinamide takes the form of a preservative-free 99% pure white crystalline powder readily soluble in water, alcohol or glycerol.

α-arbutin (INCI nomenclature: alpha-arbutin) is marketed by DSM and corresponds to the formula:

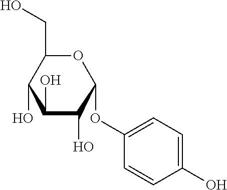

This molecule is produced by enzymatic glycosylation of hydroquinone in the presence of α-amylase and dextrin. This configuration improves its effectiveness. Its lightening activity results from direct inhibition of tyrosinase by its perfect affinity with the active site of the enzyme, unlike other arbutin derivatives such as β-arbutin (Kazuhisa Sugimoto et al. Chem. Pharm. Bull. 51 (7) 798-801 2003). Thanks to this configuration it cannot be the substrate of β-glucosidases limiting hydroquinone release. The α-arbutin takes the form of a preservative-free white powder with a content greater than 97%. This powder is readily soluble in water.

3-O-ethyl-L-ascorbic acid is a stabilized vitamin C derivative (INCI nomenclature: Ethyl-L-ascorbic acid) marketed by CosMol Co. Ltd under the name COS-VCE®. The esterification of the ascorbic acid in position 3 protects the vitamin C from degradation by high temperatures, pH, or other degradation mechanisms. 3-O-ethyl-L-ascorbic acid is more stable than ascorbic acid and its other derivatives over a wide pH range from pH 4 to pH 6, which is the usual pH range for the formulation of lightening products. 3-O-ethyl-L-ascorbic acid takes the form of a preservative-free white powder with a content greater than 98%. 3-O-ethyl-L-ascorbic acid is soluble in water and alcohol and has the following formula:

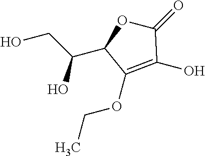

The inventors have thus demonstrated that the combination of an effective amount of the above six components acts in combination to inhibit melanogenesis in skin.

In a preferred embodiment of the invention, the concentrations of the effective amounts of the particular combination of the six components are as follows, expressed by weight relative to the total weight of the composition:

a) 0.001 to 1% of a root extract of Glycirrizha glabra,

b) 0.1 to 10% undecylenoyl phenylalanine,

c) 0.01 to 2% resveratrol,

d) 0.5 to 10% niacinamide,

e) 0.1 to 10% α-arbutin,

f) 0.5 to 20% 3-O-ethyl-L-ascorbic acid.

In a very preferred embodiment of the invention, the concentrations of the effective amounts of the particular combination of the six components are as follows, expressed by weight relative to the total weight of the composition:

a) 0.01 to 0.5% of a root extract of Glycirrizha glabra,

b) 0.5 to 4% undecylenoyl phenylalanine,

c) 0.05 to 1% resveratrol,

d) 2 to 6% niacinamide,

e) 1 to 5% α-arbutin,

f) 1 to 5% 3-O-ethyl-L-ascorbic acid.

As demonstrated in examples 5 and 6, the dermatological and cosmetic compositions according to the invention exhibit a higher activity on surface pigmentation and tissue melanin production compared to the compositions of the prior art. The inventors have discovered that the effective amounts indicated for the particular combination of these six depigmenting components solve the problem of the compositions of the prior art.

The dermatological compositions according to the invention preferably comprise the following concentrations of the six components:

a) 0.1 to 0.5% of a root extract of Glycirrizha glabra,

b) 2 to 4% of undecylenoyl phenylalanine,

c) 0.2 to 1% resveratrol,

d) 4 to 6% niacinamide,

e) 2 to 5% α-arbutin,

f) 2 to 5% 3-O-ethyl-L-ascorbic acid.

For a dermatological application, the number of applications will preferably be at least every evening for a period of 6 to 9 months.

The cosmetic compositions according to the invention preferably comprise the following concentrations of the six components:

a) 0.01 to 0.1% of a root extract of Glycirrizha glabra,

b) 0.5 to 2% undecylenoyl phenylalanine,

c) 0.05 to 0.2% resveratrol,

d) 2 to 4% niacinamide,

e) 1 to 2% α-arbutin,

f) 1 to 2% 3-O-ethyl-L-ascorbic acid.

For a cosmetic application, the number of applications will preferably be at least three applications per week for a period of two months.

In a preferred embodiment, the depigmenting dermatological or cosmetic compositions according to the invention may include other constituents or ingredients or adjuvants or additives such as especially exfoliating or keratolytic components, vitamin A, stabilized vitamin C, one or more anti-inflammatory ingredients, an organic sunscreen ingredient and/or an inorganic sunscreen agent.

Exfoliating or keratolytic ingredients include any ingredient which facilitates exfoliation such as α-hydroxy acids such as glycolic, lactic, mandelic, citric, tartaric or malic acid and β-hydroxy acids, particularly salicylic acid and its derivatives.

Vitamin A includes retinol and its different forms, retinyl palmitate, etc . . . .

Vitamin C includes different stabilized forms of vitamin C such as magnesium ascorbyl phosphate, sodium ascorbyl phosphate, ascorbyl glucoside, ascorbic acid and tetrahexyldecyl ascorb ate.

Anti-inflammatory or soothing ingredients include 18 beta glycyrrhetinic acid and bisabobol.

By sunscreen ingredient is meant a UVB or combined UVA and UVB hydrophilic organic filter, at least one UVB or combined UVA and UVB lipophilic organic filter and at least one inorganic filter.

The filters can be selected from homosalate, bis-ethylhexyloxy-phenol methoxy-phenyl triazine and polymethyl-methacrylate, ethylhexyl methoxycinnamate, tris-biphenyl triazine, diethylamino hydroxybenzoyl hexyl benzoate, diethyhexyl butamino triazone, disodium phenyl dibenzimidazole tetrasulfonate, ethylhexyl triazone, octocrylene, ethylhexyl dimethyl PABA, isoamyl p-methoxycinnamate, zinc oxide and triethoxycaprylyl-silane, phenylbenzimidazole sulphuric acid, benzophenone-3, methylene bis-benzotriazolyl tetramethylbutylphenol, ethylhexyl salicylate, butyl methoxydibenzoyl methane, titanium dioxide and zinc oxide.

In a preferred embodiment of the invention, at least one filter is selected from Zinc oxide and its derivatives, titanium dioxide, ethylhexyl salicylate, phenylbenzimidazole sulphuric acid, ethylhexyl methoxycinnamate, butyl methoxydibenzoyl methane and octocrylene.

In the compositions according to the invention, when the sunscreen ingredients are present, they are present in a level ranging from 1 to 30% by weight relative to the total weight of the composition and preferably from 5 to 25%. These sunscreen ingredients will preferably be present in the dermatological compositions according to the invention to protect the skin from the sun. They may also be present in cosmetic compositions unless the recommended application is done in the evening only.

The compositions according to the invention may also contain other conventional ingredients used in cosmetics or dermatology, such as humectants, preservatives, dyes, fragrances, penetrating agents such as diethylene glycol monoethyl ether, etc.

The compositions according to the invention will comprise a suitable carrier or excipients suitable for external topical administration, in particular dermatologically and cosmetologically acceptable excipients. These excipients suitable for the formulation are well known to those skilled in the art and include in particular penetrating agents such as ethoxydiphenol, phytantriol, octyldodecanol and escin; thickeners such as natural gums and synthetic polymers; emollients and surfactants such as cetearyl octanoate, isopropyl myristate, cetearyl isononanoate, dimethicone, cyclomethicone, polyglyceryl 3-diisostearate, hydrogenated polyisobutene, cetyl alcohol, cetyl palmitate, cetyl phosphate and capric and caprylic acid triglycerides; emulsifiers such as sorbitan esters, stearic or palmitic acid derivatives, sucroesters or glucolipids such as myristyl glucoside or stearyl glucoside; preservatives such as phenoxyethanol, methyl parahydroxybenzoate (methylparaben), ethyl parahydroxybenzoate (ethylparaben), propyl parahydroxybenzoate (propylparaben) and Phenonip® combining phenoxyethanol and methyl, ethyl, butyl and isobutyl parahydroxybenzoates; an antimicrobially active combination of a lipoamino acid, a hydroxylated fatty acid with 8 to 12 carbon atoms and glycerin alkyl ether; dyes; scents; etc.

The compositions according to the invention are present in all the galenic forms normally used in the cosmetic and dermatological fields which are suitable for topical application, such as, for example: a serum lotion, an emulsion of more or less fluid consistency, white or colored, obtained by dispersing a fatty phase in an aqueous phase, an oil-in-water (O/W) emulsion or conversely a water-in-oil (W/O) emulsion such as a milk, cream, gel or gel-emulsion, a mask or an anhydrous balm, oil or powder product. They may also be in stick form.

Among these preparations, the preference is for creams, fluid or thick gels or sprays, which can be simply and easily applied on all parts of the body.

The invention also relates to a process for preparing the compositions described above by mixing, according to conventional methods, the active ingredients with the carrier and the other ingredients which may be present.

The compositions according to the invention are topically applied in sufficient quantity for humans, i.e. in an amount corresponding to the usual application rates for the type of composition in question (gel, cream, lotion, etc.). For example, in the case of a cream, we use 0.5 to 3 mg and in particular 1 to 2 mg of cream per cm² of skin per application, at a rate of one to five applications per day depending on the desired treatment.

The invention further relates to the particular use of the following six components acting in combination on melanogenesis,

a) a root extract of Glycirrizha glabra,

b) undecylenoyl phenylalanine,

c) resveratrol,

d) niacinamide,

e) α-arbutin,

f) 3-O ethyl-L-ascorbic acid,

as active compounds for the preparation of a dermatological composition intended for the treatment of pathological or accidental hyperpigmentations.

In a preferred embodiment, the above use is made with a dermatological composition according to the invention comprising the following concentrations of the six components:

a) 0.1 to 0.5% of a root extract of Glycirrizha glabra,

b) 2 to 4% undecylenoyl phenylalanine,

c) 0.2 to 1% resveratrol,

d) 4 to 6% niacinamide,

e) 2 to 5% α-arbutin,

f) 2 to 5% 3-O ethyl-L-ascorbic acid.

In a preferred embodiment of the invention, the dermatological use of the six components for application on the skin is carried out at one to two applications per day for at least six months to obtain a lasting depigmentation of the epidermis.

The invention further relates to a cosmetic treatment process to improve the aesthetic appearance of the skin and consists in applying to the skin a sufficient amount of a composition according to the invention.

In a preferred embodiment, in the method of cosmetic treatment according to the invention, the application to the skin is carried out at one application per day, preferably in the evening, for two months to obtain a lasting depigmentation of the epidermis.

The invention and the advantages thereof will be better understood from reading the description and non-limiting embodiments which follow, written in light of the accompanying figures in which:

Figure A (example 6) shows the change in surface pigmentation in the in vitro test described, expressed as a percentage of Optical Density (OD) at 492 nm between T0 and T+10 days.

Figure B (example 6) shows the change in the amount of tissue melanin in the in vitro test described, expressed as a percentage between T0 and T+10 days.

Figure C (example 7) shows the skin melanin index in the in vivo test in example 7.

### EXAMPLE 1: LIGHTENING FACE CREAM

The procedure to prepare the cream is as follows:

The different phases are weighed.

Phase A is heated to 70° C.

Phase B is heated to 70° C. and xantham gum is added with stirring until a homogeneous system is obtained.

Phase A is poured into B with rapid stirring until a homogeneous system is obtained.

The mixture is allowed to cool to room temperature with stirring.

Phases C and D are then added with stirring.

| INCI name* | % p/p |
|---|---|
| PHASE A | |
| Glyceril stearate (and) PEG-100 stearate | 3 to 10% |
| Cetearyl alcohol | 0.5 to 2% |
| Octyldodecanol | 1 to 5% |
| Olea europaea (olive) extract | 0.5 to 4% |
| Dicaprylyl ether | 1 to 5% |
| Glycirrizha glabra (licorice) root extract | 0.01 to 0.1% |
| Undecylenoyl phenylalanine | 0.5 to 2% |
| Resveratrol | 0.05 to 0.2% |
| Dimethicone | 0.5 to 3% |
| PHASE B | |
| Aqua | QS |
| Disodium EDTA | 0.05 to 0.5% |
| Xanthan Gum | 0.1 to 2% |
| Gluconolactone (and) sodium benzoate (and) calcium | 0.5 to 2% |
| gluconate | |
| PHASE C | |
| Aqua | 4 to 8% |
| Niacinamide | 2 to 4% |
| Alpha-Arbutin | 1 to 2% |
| 3-O-Ethyl-Ascorbic acid | 1 to 2% |
| PHASE D | |
| Preservatives | 0.1 to 2% |

*INCI: International Nomenclature of Cosmetic Ingredients

### EXAMPLE 2: LIGHTENING BODY LOTION

The procedure to prepare the mask is as follows:

The different phases are weighed.

Phase A is heated to 70° C.

Phase B is heated to 70° C.

Phase A is poured into B with rapid stirring until a homogeneous system is obtained.

The mixture is allowed to cool to room temperature with stirring.

Lastly, phases C and D are added with stirring.

| INCI name* | % p/p |
|---|---|
| PHASE A | |
| Potassium palmitoyl, hydrolyzed wheat protein, glyceryl | 2 to 10% |
| stearate, cetearyl alcohol | |
| Cetearyl alcohol | 0.5 to 2% |
| Prunus amigdalus dulcis oil | 2 to 5% |
| Coco-caprylate/caprate | 0.5 to 3% |
| Glycirrizha glabra (licorice) root extract | 0.01 to 0.1% |
| Undecylenoyl phenylalanine | 0.5 to 2% |
| Resveratrol | 0.05 to 0.2% |
| Tocopheryl acetate | 0.05 to 0.5% |
| Dimethicone | 0.5 to 3% |
| PHASE B | |
| Aqua | QS |
| Glycerin | 0.5 to 3% |
| Disodium EDTA | 0.05 to 0.5% |
| Carbomer | 0.1 to 1% |
| PHASE C | |
| Aqua | 4 to 8% |
| Niacinamide | 2 to 4% |
| Alpha-Arbutin | 1 to 2% |
| 3-O-Ethyl-Ascorbic acid | 1 to 2% |
| PHASE D | |
| Preservatives | 0.1 to 2% |

### EXAMPLE 3: LIGHTENING FACE MASK

The procedure is the same as in example 2.

| INCI name* | % p/p |
|---|---|
| PHASE A | |
| stearate (and) PEG-100 stearate A | 3 to 9% |
| Cetearyl alcohol | 0.5 to 2% |
| Prunus amigdalus dulcis oil | 2 to 5% |
| Heliantuus annuus seed oil | 0.5 to 4% |
| Dicaprylyl ether | 1 to 5% |
| Glycirrizha glabra (licorice) root extract | 0.01 to 0.1% |
| Undecylenoyl phenylalanine | 0.5 to 2% |
| Resveratrol | 0.05 to 0.2% |
| Tocopheryl acetate | 0.05 to 0.5% |
| Dimethicone | 0.5 to 3% |
| PHASE B | |
| Aqua | QS |
| Glycerin | 0.5 to 3% |
| Disodium EDTA | 0.05 to 0.5% |
| Carbomer | 0.1 to 1% |
| PHASE C | |
| Aqua | 4 to 8% |
| Niacinamide | 2 to 4% |
| Alpha-Arbutin | 1 to 2% |
| 3-O-Ethyl Ascorbic acid | 1 to 2% |
| PHASE D | |
| Preservatives | 0.1 to 2% |

### EXAMPLE 4: LIGHTENING SERUM

The procedure is the same as in example 2.

| INCI name* | % p/p |
|---|---|
| PHASE A | |
| Dicaprylyl ether | 1 to 5% |
| Caprilic capric triglycerides | 1 to 5% |
| Isononil isononanoate | 0.5 to 5% |
| Glycirrizha glabra (licorice) root extract | 0.01 to 0.1% |
| Undecylenoyl phenylalanine | 0.5 to 2% |
| Resveratrol | 0.05 to 0.2% |
| Tocopheryl acetate | 0.05 to 0.5% |
| PHASE B | |
| Aqua | QS |
| Glycerin | 0.5 to 3% |
| Disodium EDTA | 0.05 to 0.5% |
| Carbomer | |
| Acrylates/C10-30 alkyl acrylate crosspolymer | 0.01 to 1% |
| PHASE C | |
| Aqua | 4 to 8% |
| Niacinamide | 2 to 4% |
| Alpha-Arbutin | 1 to 2% |
| 3-O-Ethyl-Ascorbic acid | 1 to 2% |
| Preservatives | 0.1 to 2% |

### EXAMPLE 5: PROTECTIVE LIGHTENING CREAM SPF 30

The procedure is the same as in example 1.

| INCI name* | % p/p |
|---|---|
| PHASE A | |
| Polyglyceryl-6 distearate (and) jojoba esters (and) | 3 to 9% |
| polyglyceryl-3 beeswax (and) cetyl alcohol | |
| Cetearyl alcohol | 0.5 to 2% |
| Coco- caprylate | 0.5 to 2% |
| Ethylhexyl palmitate | 0.5 to 3% |
| Butyl methoxydibenzoylmethane | 4 to 8% |
| Ethylhexyl salicylate | 3 to 8% |
| Homosalate | 5 to 10% |
| Ehtylhexyl methoxycinnamate | 2 to 8% |
| Octocrylene | 2 to 10% |
| Glycirrizha glabra (licorice) root extract | 0.01 to 0.1% |
| Undecylenoyl phenylalanine | 0.5 to 2% |
| Resveratrol | 0.05 to 0.2% |
| Tocopheryl acetate | 0.05 to 0.5% |
| Dimethicone | 0.5 to 3% |
| PHASE B | |
| Aqua | QS |
| Glycerin | 0.5 to 3% |
| Disodium EDTA | 0.05 to 0.5% |
| Xanthan gum | 0.1 to 2% |
| PHASE C | |
| Aqua | 4 to 8% |
| Niacinamide | 2 to 4% |
| Alpha-Arbutin | 1 to 2% |
| 3-O-Ethyl Ascorbic acid | 1 to 2% |
| PHASE D | |
| Preservatives | 0.1 to 2% |

### EXAMPLE 6: EVALUATION OF THE IN VITRO DEPIGMENTING EFFECT OF A CREAM ACCORDING TO THE INVENTION

The depigmenting effect was evaluated in vitro on 10-day-old phototype VI pigmented reconstructed human epidermises.

The lightening face cream described in example 1 is the formula used in the in vitro test on a reconstituted epidermis.

Reconstructed Human Tissues

The pigmented epidermis model is rebuilt from primary cultures of keratinocytes and melanocytes using a reconstruction process at the air-liquid interface on a defined medium.

Methodology

The test elements were placed in contact on the reconstructed tissues for 10 days by topical application for 45 minutes at a time for 10 days. For each condition, the tissues are placed in contact with a single quantity of test element and are incubated at 36.5° C./5% CO₂.

After 10 days of treatment, three tissues per condition are used for the melanin quantification. The three tissues are used beforehand for the measurement of the surface melanin index.

Equipment

Four compositions were prepared to perform the various tests in this evaluation. The common base of three of the four compositions is as follows:

Formulation Base

| INCI name* | % p/p |
|---|---|
| PHASE A | |
| Glyceril stearate (and) PEG-100 stearate | 3 to 15% |
| Cetearyl alcohol | 0.5 to 5% |
| Octyldodecanol | 1 to 5% |
| Olea europaea (olive) extract | 0.5 to 5% |
| Dicaprylyl ether | 0.5 to 5% |
| Dimethicone | 0.5 to 5% |
| PHASE B | |
| Aqua | QS |
| Disodium EDTA | 0.05 to 0.5% |
| Xanthan Gum | 0.1 to 2% |
| Gluconolactone (and) sodium benzoate (and) calcium | 0.5 to 3% |
| gluconate | |

The four compositions tested are:
A composition according to the invention corresponding to the cream in example 1 above.
1% kojic acid: This composition corresponds to the positive control. It comprises a cream consisting of the common base and 1% kojic acid.
A cosmetic reference: This is the cosmetics market reference in the results below. It corresponds to a composition containing 4% kojic acid, 2% magnesium ascorbyl phosphate and 2% niacinamide together with 0.1% of the root extract of Glycirrizha glabra and the common base.
A dermatological reference: the cream named “KLIGMAN trio” is the dispenser's preparation manufactured by the Delpech Fabre laboratories and is formulated with 4% hydroquinone, 0.1% dexamethasone acetate, 0.03% retinoic acid and the base NOURIVAN™ ANTIOX (QSP).

Culture Medium:

The culture medium 1610EM068 used in this test is a specific medium for the reconstruction of pigmented skins: it contains all the necessary growth factors for primary keratinocytes and melanocytes.

Test Protocol

a) Application of the Products on the Reconstructed Tissues

The tissues are treated for 10 days for 45 minutes at a time at 36.5° C./5% CO₂.

For the negative control, the tissues are incubated in the culture medium.

Each condition is produced on four tissues and all the tissues are placed in 1 ml of medium per day.

b) Rinsing of the Reconstructed Tissues

After 15 minutes of incubation at 36.5° C./5% CO₂, the tissues are rinsed with 10 ml of phosphate buffer solution (PBS) and allowed to stand until the next application.

c) Measurement of Surface Pigmentation

The measurement of the surface pigmentation of the tissues is made using the Mexameter MX18 on the four tissues in each condition.

d) MTT Viability Test

After 10 days of treatment one tissue per condition is used to check the viability. They are placed in 300 μl of MTT solution at 0.5 mg/ml: incubation for 3 hours at 36.5° C./5% CO₂.

Each tissue is transferred to a well containing 1.5 ml of isopropanol: incubation for at least 1 hour at room temperature. After incubation, the tissues are removed from the wells.

After homogenization, 200 μl from each well are transferred in triplicate to a 96-well plate and an absorbance reading is made at 550 nm.

For each condition, the ratio of the mean optical density of the tissues to the mean optical density of the negative controls determines the viability rate.

d) Melanin Assay

The melanin is quantified by colorimetric assay on three tissues per condition.

The tissues are placed in 400 μl of Perkin Elmer Solvable™ solution.

The tissue melanin is hot-extracted for 1 hour at 100° C.

After extraction, an absorbance reading is made at 492 nm.

For each condition, the melanin concentration is calculated using the melanin standard curve.

Results
The quality of the tissues used for the study meets the criteria: at D10, the pigmented epidermises present an OD greater than 0.8 (OD=1.333) and good tissue cohesion, and the presence of melanin is confirmed by the Fontana Masson stain.
The change in surface pigmentation in the in vitro test is expressed as a percentage of Optical Density (OD) at 492 nm between T0 and T+10 days in Figure A.
The negative control (untreated) confirms an increase in surface pigmentation of 28% in 10 days. The positive control (1% kojic acid) confirms a slowing of the surface pigmentation with an increase of 18%. These expected results validate the test.

As regards the products tested, the lightening composition according to the invention showed the best efficiency in slowing surface pigmentation with an increase of only 3% after 10 days of treatment. This efficiency is greater than that of both the cosmetic reference and the dermatological reference, 20% and 21% respectively (see Figure A).
The change in the amount of tissue melanin is expressed as a percentage between T0 and T+10 days in Figure B.
The negative control (untreated) confirms the increase in tissue melanin of 203% in 10 days. As regards the positive control (1% kojic acid), it does indeed present a slowing of melanin synthesis with an increase of 133%. These expected results validate the test (see Figure B).

In the tested products, the lightening composition according to the invention demonstrates a greater efficiency with an increase of only 35% while the cosmetic and dermatological references have an increase in tissue melanin of 107% and 80% respectively.

This test highlights the superiority of the cosmetic compositions on both parameters, surface pigmentation and production of tissue melanin. It corroborates the fact that the effective amounts of the particular combination of the six components of the compositions according to the invention act in combination on melanogenesis with a much better efficiency than kojic acid and the cosmetic and dermatological references of the prior art.

### EXAMPLE 7: EVALUATION OF THE IN VIVO DEPIGMENTING EFFECT OF A CREAM ACCORDING TO THE INVENTION

This study was conducted on a panel of 22 female volunteers aged 34 to 65, with an average age of 46, of phototype III to V according to the Fitzpatrick classification. Ten subjects had melasma, ten other subjects had lentigo and two subjects had melasma and lentigo. These volunteers followed a protocol of care combining four surface chemical peels performed by a professional at regular 15-day intervals followed by application in the evening of the cream, the composition of which is described below, for a period of 56 days. During the day an SPF 15 sunscreen combined with a moisturizing emulsion is applied.

| INCI name* | % p/p |
|---|---|
| AQUA | QS |
| 3-O-ETHYL ASCORBIC ACID | 2 to 5% |
| ALPHA-ARBUTIN | 2 to 5% |
| AMINOMETHYL PROPANOL | 0.1 to 2% |
| CETEARYL ALCOHOL | 0.5 to 3% |
| CYCLOPENTASILOXANE | 2 to 8% |
| DIMETHICONE | 0.5 to 3% |
| DISODIUM EDTA | 0.05 to 1% |
| GLUCONOLACTONE | 0.5 to 3% |
| GLYCERYL STEARATE | 3 to 10% |
| GLYCYRRHETINIC ACID | 0.1 to 2% |
| GLYCYRRHIZA GLABRA (LICORICE) ROOT EXTRACT | 0.1 to 0.5% |
| LECITHIN | 0.5 to 3% |
| MANDELIC ACID | 5 to 15% |
| NIACINAMIDE | 4 to 6% |
| OCTYLDODECANOL | 1 to 5% |
| OLEA EUROPAEA OIL UNSAPONIFIABLES | 0.5 to 4% |
| PEG-100 STEARATE | 3 to 10% |
| POLYSORBATE 20 | 0.05 to 1% |
| PROPYLENE GLYCOL | 0.5 to 3% |
| RESVERATROL | 0.2 to 1% |
| RETINOL | 0.05 to 1% |
| SALICYLIC ACID | 1 to 5% |
| SODIUM BENZOATE | 0.1 to 2% |
| UNDECYLENOYL PHENYLALANINE | 2 to 4% |
| XANTHAN GUM | 0.1 to 2% |

Experimental Data:

Measurements made with a Mexameter highlighted a very significant average melanin index reduction of 9.2% at 28 days (p<0.001) and 24.2% at 56 days (p<0.00001) with a maximum reduction of 40.2% at 56 days. These results are presented in Figure C. No serious adverse reactions are noted and 81% of patients showed good to excellent tolerance of the treatment.

These results demonstrate the effectiveness of the compositions according to the invention and their good tolerance.

1. A dermatological or cosmetic composition having a depigmenting action by topical application and comprising at least an effective amount of a particular combination of the following six components acting in combination on melanogenesis:
a) a root extract of Glycirrizha glabra,
b) undecylenoyl phenylalanine,
c) resveratrol,
d) niacinamide,
e) α-arbutin,
f) 3-O ethyl-L-ascorbic acid,
in a carrier suitable for such an application.

2. A composition according to claim 1, wherein the concentrations of the six components are as follows, expressed by weight relative to the total weight of the composition:
a) 0.001 to 1% of a root extract of Glycirrizha glabra,
b) 0.1 to 10% undecylenoyl phenylalanine,
c) 0.01 to 2% resveratrol,
d) 0.5 to 10% niacinamide,
e) 0.1 to 10% α-arbutin,
f) 0.5 to 20% 3-O ethyl-L-ascorbic acid.

3. The composition according to claim 2, wherein the concentrations of the six components are as follows, expressed by weight relative to the total weight of the composition:
a) 0.01 to 0.5% of a root extract of Glycirrizha glabra,
b) 0.5 to 4% undecylenoyl phenylalanine,
c) 0.05 to 1% resveratrol,
d) 2 to 6% niacinamide,
e) 1 to 5% α-arbutin,
f) 1 to 5% 3-O ethyl-L-ascorbic acid.

4. The composition according toe claim 1, wherein the composition additionally comprises exfoliating components, vitamin A, stabilized vitamin C and anti-inflammatory components, one or more anti-inflammatory ingredients, an organic sunscreen ingredient and/or an inorganic sunscreen ingredient.

5. The composition according to claim 1, wherein the composition takes the form of a cream, fluid or thick gel or spray.

6. The composition according to claim 1, wherein an effective amount of a particular combination of the following six components acting in combination on melanogenesis:
a) a root extract of Glycirrizha glabra,
b) undecylenoyl phenylalanine,
c) resveratrol,
d) niacinamide,
e) α-arbutin,
f) 3-O ethyl-L-ascorbic acid,
as active compounds for the preparation of a dermatological composition intended for the treatment of pathological or accidental hyperpigmentations.

7. The composition according to claim 6, for application on the skin at a rate of one to two applications per day for six months to obtain a lasting depigmentation of the epidermis.

8. A cosmetic treatment process to improve the aesthetic appearance of the skin, consists in applying to the skin a sufficient amount of a composition according claim 1.

9. The cosmetic treatment process according to claim 8, for application on the skin at a rate of one application per day, preferably in the evening, for two months, to obtain a lasting depigmentation of the epidermis.
